(19) United States
(12) Patent Application Publication
(10) Pub. No.: US 2003/0166010 A1
(43) Pub. Date: Sep. 4, 2003

(54) Custom ligand design for biomolecular filtration and purification for bioseperation

(76) Inventor: Joseph A. Affholter, Coleman, MI (US)

Correspondence Address: Martha Finnegan, Esq., CABOT CORPORATION, 157 Concord Road, P.O. Box 7001, Billerica, MA, 01821-7001, US

(21) Appl. No.: 10/374,817

(22) Filed: Feb. 25, 2003

Related U.S. Application Data
(60) Provisional application No. 60/359,503, filed on Feb. 25, 2002.

(51) Int. Cl. G01N 33/53; G01N 33/543

(52) U.S. Cl. 435/7.1; 436/518

(57) ABSTRACT

The instant invention relates to a method of isolating a bio-active molecule of interest from a biological system using affinity separation. The affinity separation ligand for the bio-active molecule of interest is selected by directed molecular evolution to customize the properties of the interaction between the bio-active molecule of interest and the affinity separation ligand thereby facilitating isolation of the bio-active molecule of interest.

The invention also relates to compositions comprising an affinity separation ligand bound to a solid support wherein the separation ligand is selected by the method of directed molecular evolution.

### DESCRIPTION OF THE INVENTION

1. Field of the Invention

The invention relates generally to the field of bio-molecular separation or purification using affinity ligands. More specifically, the invention relates to protein and/or peptide separation or purification using affinity ligands.

2. Background of the Invention

The use of biological systems e.g. prokaryotic or eukaryotic cells as a source of, or means of production of, bio-active molecules creates the need for methods to separate or isolate the bio-active molecule of interest. Biological systems are a complex mixture of organic and inorganic compounds. Many of these compounds have potentially deleterious effects on the activity or stability of the bio-active molecule of interest. Moreover, the stability requirement of proteins in a cell are not ideal for use in a non-cellular environment (e.g. separation). Therefore, selecting for stability of a purified/separated molecule of interest is important in identifying bio-active molecules compatible with bio-separation. Purification and or separation therefore is an essential step in the production of any biologically derived molecule. To preserve the activity and stability of a bio-active molecule of interest the separation from other components of the system must be both rapid and effective.

Typically, the molecule of interest is a protein or peptide. Separation or purification of a protein can be achieved by a variety of means known in the art, e.g. chromatography, which exploit chemical or physical properties of the protein such as size, solubility, charge or specific binding affinity. Affinity separation is a method of separating or isolating molecules. While not limited to biological applications, it is most often applied to macromolecules of biological origin (e.g. proteins or peptides). The separation is achieved by exploiting the bio-molecule's tendency to interact with or bind to a specific ligand under certain conditions. Subsequent alteration of these conditions results in the release of the bio-molecule from its ligand. Many such conditions are known in the art. Examples of the conditions that can be altered to release the bio-molecule from its ligand include, but are not limited to, pH, salt concentration, ionic strength, detergent or denaturant concentration, addition of competitor or displacer molecules, alteration of temperature and addition, or removal, of chelants, surfactants or stabilizers.

Frequently, affinity separation relies on the use of monoclonal or polyclonal antibodies that specifically bind to a molecule of interest. Antibodies can be covalently or non-covalently bound to a solid support and the source material containing the molecule of interest is passed over the solid support. The molecule of interest binds specifically to the antibody and is retained on the solid support. Lowering the pH results in the elution of the bound molecule of interest.

Antibodies provide high specificity and tight ligand binding and thus would seem like ideal candidates for use in affinity separation, however, development of monoclonal or polyclonal antibodies can be both time consuming and expensive. Furthermore, since polyclonal and monoclonal antibodies are typically generated by immunizing living animals (typically rabbits, or mice) the repertoire of epitopes that can be recognized by any given antibody is limited by the genetic background (at the VDJ loci) of the immunized animal. Additionally, the coupling chemistry, elution, regenerating or cleaning conditions can adversely affect stability of antibodies on the support.

Other (e.g. non-antibody) classes of polypeptides can also be ideal choices as ligands for purifying certain bioactive polypeptide therapeutics. For example, when a given class of cell polypeptide receptors, adhesion molecules, enzymes or other biochemicals are known to bind specifically to the bioactive substance of interest, such binding agents may provide ideal starting activity from which to develop an improved affinity separation ligand or matrix. For example, one might use genetically engineered, soluble (e.g. secreted or membrane unassociated) versions of a tumor necrosis factor, interleukin or colony stimulating factor as starting agents for developing an affinity separation matrix for purifying the bioactive factors known to bind them. While this is theoretically possible, the complexities of working with unstable, poorly expressed low solubility receptors limit the practical utlility of such polypeptides as affinity ligands. As biotherapeutics developers begin developing second generation protein therapeutics, it will become increasingly important to generate new affinity ligands and separation strategies optimized for the new rather than wild type bioactive agents.

Rapid advances in reiterative diversification and screening technologies (e.g. directed molecular evolution) are making it increasingly possible to rapidly generate, identify and produce proteins optimized with defined performance properties.

Directed evolution or in vitro evolution is a recombinant DNA and/or synthetic nucleic acid technology that provides for the rapid selection of proteins or peptides with a desired characteristic or phenotype. For example proteins can be selected that are stable over a wide range of temperatures, pH or salt concentration (see e.g. U.S. Pat. Nos. 5,830,696, 5,935,920). The potential to automate both the diversity and the screening methodologies of directed evolution is also known in the art (see e.g. U.S. patent application Ser. No. 09/760,010). Several forms of directed evolution mimic the laborious, linear, mutagenesis-based methods for sequence improvement employed by most prokaryotic organisms. While other methods mimic the recombination-based diversity generation methods used by higher organisms to generate and test genetic diversity (see e.g. U.S. Pat. Nos. 6,337,186, 6,372,497, 6,297,053, 5,605,793, 6,277,638, 6,117,679, 6,303,344, 6,319,714, 6,335,166, 6,177,263, 6,153,410 and U.S. application Ser. No. 09/760,010).

One class of methods which mimic diversity generation methods found in eukaryotes, most commonly referred to as DNA shuffling, gene shuffling or gene reassembly, can be referred to as molecular breeding or more generically as recombination methods. The result of mimicking sexual reproduction is dramatically increased efficiency in the performance improvement process. In vivo forms of molecular breeding are known and used in the art (e.g., whole genome shuffling). However, in molecular breeding methods, gene recombination is generally conducted in an in vitro setting, thus allowing for exquisite control of the resultant phenotype.

Molecular breeding involves the in vitro combination and recombination of genetic homologs that share genetic sequence identity over a range of nucleotides. The range of nucleotides can vary from a small number of nucleotides to a long stretch of nucleotides. The number of parental genes or gene fragments may range from one to a large number. In some examples known in the art >25 individual parents can contribute to a recombination mixture. While there is no apparent upper limit to the number of parents that can contribute to developing a high performance gene through recombination technology, practical limits may emerge based only on sample handling capacity or the length of the target sequence. Typically, parental genes are subjected to fragmentation by one of many methods (e.g. enzymatic, synthetic oligonucleotide-based, etc.). The addition of a polymerase (e.g. Taq polymerase, thermophilic DNA polymerases or other polymerases, such as the Klenow fragment of E. coli DNA polymerase I) and multiple cycles of denaturation and polynucleotide cross-priming and extension generates newly recombined mutants. The proteins expressed from these recombined homologs can be expressed and the desired phenotypic properties of the protein can be screened for. The process of recombination, expression and screening can be repeated several times to improve upon the desired phenotypic trait of the protein (see e.g. U.S. Pat. No. 6,337,186).

In addition to molecular breeding technologies, a variety of random and directed mutagenesis technologies have also been developed to achieve the goals of directed molecular evolution or reiterative diversification. Some examples of these technologies include error prone PCR (Caldwell et al., 1992 PCR Methods Applic., 2:228) cassette mutagenesis, site directed mutagenesis, recursive ensemble mutagenesis, (Goldman et al., 1992 Nature Biotechnology 10: 1557) and in vitro scanning saturation mutagenesis (U.S. Pat. No. 6,180,341). Molecular breeding allows one to interchange cycles of mutagenesis with recombination, or to run both processes simultaneously.

Cassette mutagenesis involves mutating specific target DNA sequences. The target DNA sequences, for example, can code for amino acids or control sequences (e.g., promoters, enhancers). A mutagenic oligonucleotide cassette(s) is (are) generated to introduce changes into the target site. A group of amino acids can be targeted, for example, based upon their predicted location in the three dimensional structure of the protein, statistical or homology-based relationships, the positions of observed variations within aligned groups of homologous sequences and other similar strategies. The mutants are expressed and the desired properties can then be screened for.

Scanning saturation mutagenesis targets individual amino acids for mutation. Typically, individual amino acids within a given region or a subset of amino acid positions are subjected to mutation to create a population in which each progeny polynucleotide encodes a protein having a single amino acid alteration from the parental sequence. The mutants are expressed and the desired properties are screened for.

Other methods of reiterative diversification technologies which permit the mutagenesis and/or evolution of a DNA sequence encoding a protein of interest, or fragment thereof, are known in the art. These include, for example, oligonucleotide directed mutagenesis (Reidhaar-Olson et al. 1988, Science 241:53), exponential ensemble mutagenesis (Delegrave et al. 1993, Biotechnology Research 11:1548), and a variety of related methodologies that have been described (e.g., U.S. Pat. Nos. 5,223,408, 5,264,563, 5,279,952, 5,314,809, 5,316,935, 5,512,463, 5,514,568, 5,521,077, 5,523,388, 5,605,793, 5,698,426, 5,708,153, 5,723,323, 5,763,192, 5,770,434, 5,808,022, 5,811,238, 5,814,476, 5,830,721, 5,837,458, 5,871,974, 5,928,905, 5,939,250, 5,955,358, 5,958,672, 5,965,408, 5,976,862, 6,001,574, 6,030,779, 6,054,267, 6,057,103, 6,071,889, 6,096,548, 6,117,679, 6,132,970, 6,153,410, 6,159,687, 6,168,919, 6,177,263, 6,180,406, 6,238,884, 6,280,926, 6,291,158, 6,291,161, 6,291,242, 6,297,053, 6,303,344, 5,348,867, and 5,866,344).

In addition, a variety of multi-cycle adsorption-based screening (MCAS) technologies are also known in the art. MCAS technologies include, for example, bacteriophage display strategies (U.S. Pat. Nos. 5,223,409, 5,403,484), ribosomal display (Mattheakis et al. 1994, Proc. Natl. Acad. Sci. USA 91:9022) polysome display (U.S. Pat. No. 5,643,768) and yeast and bacterial display technologies. They also include methods that specifically link a nucleic acid sequence to a protein or protein fragment encoded thereby, and thus permit the isolation of said nucleic acid sequence encoding said protein or protein fragment, (See e.g. U.S. Pat. Nos. 6,312,927, 6,057,103, and PCT Publication No. WO 01/05808). It can be particularly useful to combine MCAS with reiterative diversification technologies thus allowing for the screening of a large number of generated recombinants.

Another aspect of affinity separation is the need to select a solid support which is attached to the desired affinity ligand. Selection of the appropriate solid support and method of linking the desired affinity ligand to the solid support should be optimized in conjunction with selection of the desired ligand. Thus, while screening of affinity ligands using phage display has been described (U.S. Pat. No. 326,155), linking affinity ligands to a solid support such that the desired trait in the ligand is maintained or enhanced has not.

The invention described herein addresses the problems associated with polypeptide based affinity separation by combining the technology of reiterative diversification and screening with affinity separation to provide for a customized method of purifying biological molecules of interest, such as proteins or peptides, that is rapid, inexpensive, safe and effective The instant invention, includes screening a plurality of input parameters to define performance, stability and/or selectivity screens that are then used to develop custom combinations of ligand, linking chemistry and support material which provide an affinity separation matrix with optimized specified properties.

### SUMMARY OF THE INVENTION

Reiterative diversitification and screening is used to generate affinity ligands that possess a desired phenotype or characteristic e.g. bind with high affinity to a molecule of interest. The ligand is then bound or linked to a solid support via a specific linker chemistry allowing for affinity separation that is tailored to the specificity of a molecule of interest. The invention, therefore, relates to a customized method of performing affinity separation whereby a ligand which specifically binds a molecule of interest and which possesses a desired phenotype or characteristic is selected using any method of directed molecular evolution or reiterative diversification and screening, and wherein said ligand is bound or linked to a solid support using specific linker chemistry. The invention further relates to compositions useful for performing customized affinity separation wherein said composition is comprised of a solid support and a ligand bound or linked via specific linker chemistry to the solid support, said ligand being produced or selected for by any method of directed molecular evolution or reiterative diversification and screening. The invention further relates to a method of isolating an affinity ligand, which possesses a desired phenotype or characteristic, using any method of directed molecular evolution or reiterative diversification and screening and linking the affinity ligand to a solid support such that the desired phenotype or characteristic is maintained or enhanced.

Description of the Embodiments

As used herein, affinity ligand means a first molecule (e.g. protein, nucleic acid, DNA, RNA, lipid, or sugar) or fragment thereof, that specifically interacts (e.g. binds) with a second molecule of interest.

The invention relates to a method of separating biological molecules of interest which are derived from, or produced in, a biological system, from other components of the biological system, using affinity separation wherein said affinity separation is comprised of the steps of (1) obtaining or generating a genetic library which encodes potential affinity ligands for the biological molecule of interest using a method of directed molecular evolution or reiterative diversification and screening; (2) expressing (or synthesizing) the affinity ligands generated or obtained in step (1) screening the expressed affinity ligands of step (2) for a desired phenotypic trait, (3) linking or binding the ligand of step (2) to a solid or semi-solid support; (4) contacting the ligand linked or bound to the solid or semi-solid support with the biological molecule of interest under conditions such that the biological molecule binds to the ligand and is retained on the support-ligand complex; (5) optionally washing the complex of step (4) with a buffer or otherwise removing molecules non-specifically bound to the affinity ligand or collecting or sorting the support-ligand complex from unabsorbed material; and (6) eluting the biological molecule of interest by changing the conditions of step (4). It will be understood by those skilled in the art that the above steps need not be performed in the recited order and that individual steps (e.g. screening) can be performed independently of other steps. It will also be understood that these steps can be repeated at least more than once to generate additional improvements or alternative ligand-support combinations.

In one embodiment reiterative diversification and screening (or directed molecular evolution) is used to identify one or more protein ligands with one or more improved performance parameters in a plurality of cycles of diversification and screening. In this embodiment isolated parental genes (e.g. nucleic acid sequences which are cloned, purified, and characterized) are used as starting material. In one embodiment the number of isolated parental genes is less than 100. In another embodiment the number of parental genes is less than 25, less than 10, less than 5. In yet another embodiment the number of parental genes is greater than 25. In one embodiment improvement in performance is measured against a starting material or standard reagent, preferably having at least a minimal activity (e.g. binding activity) in the separations applications of the invention. In another embodiment an inactive reference is used as reference material until an agent with initial activity is identified. In another embodiment the reiterative diversification and screening process identifies agents (e.g. ligands) with improvements in at least two defined performance parameters in a plurality of diversification and screening cycles. In another embodiment the reiterative diversification and screening process identifies improvements in at least one chemoselectivity parameter and chemical or physical stability parameter. In another embodiment the reiterative diversification and screening process identifies improvements in one chemoselectivity and one bioproduction parameter (e.g. yield, proper processing, transcription or translational response and control). In another embodiment the methods of the invention yield improvements in at least one physical or chemical stability parameter, and at least one bioproduction parameter in a multi-cycle sequence diversification and screening process.

Examples of physical or chemical stability parameters include, without limitation, retention of biological activity under any of the following conditions: in the presence of one or more organic solvents (e.g. at concentrations of >0.1%); at temperatures above or below normal physiological range (e.g. less than 25° C. or greater than 40° C.); at pH of <7.0 or >8.0; in salt concentrations less than 100 mM or greater than 200 mM; in the presence of detergents (e.g. anionic, cationic, nonpolar, and polymeric surfactants); in the presence of hydrogen bond breaking denaturants (e.g. guanidine hydrochloride, urea,); in the presence of >0.001% hypochlorite, sodium azide, or other agents used as sterilizers or preservatives; in the presence of high bulk or local shear forces; in the presence of mono-, bi-, or polyfunctional crosslinking agents, chemical fixing agents, alkylating agents, strong electrophiles or strong nucleophiles which might be used for linking a ligand to a solid support; in the presence of other proteins or biological materials (e.g. tissue or cellular lysate). In addition, important chemical stability parameters may include resistance to inactivation in the presence of epoxides, activated carboxylates, esters, imides, amines, imines, hydrazines, alkyl thiols and other groups commonly used for immobilizing proteins upon solid supports. (see e.g., Pierce Catalog 2002) (Pierce Biotechnology, Inc., Rockford, Ill.) It can also include resistance to inactivation upon exposure to one or more solid support materials.

Examples of chemoselectivity parameters include any physicochemical measurements intended to assess the kinetic or thermodynamic properties of the ligand:target interaction. Most commonly, this comprises methods for determining selectivity, specificity, avidity, affinity, and equilibrium constants relating to the ligand:target interaction. As additional non-limiting examples, chemoselectivity parameters also include: off-rates, on rates, association, dissociation, inhibition and other equilibrium constants (e.g. Kₘ, Kᵢ), physical adsorption, retention or retention times, luminescence, fluorescence, radioactive and electromagnetic signals indicative of specific binding interactions.

A genetic library can be obtained using methods known in the art. See e.g. Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989). In one embodiment the library is a cDNA library obtained by isolating mRNA on polyA column, reverse transcribing the RNA into DNA and cloning the DNA into an appropriate vector e.g. an expression vector. In another embodiment the library can be comprised of genomic DNA. In another embodiment the library can be derived from isolated homologs of a known protein, peptide or fragment thereof. In another embodiment the library is generated by selective DNA amplification, wherein a set of primers capable of directing amplification of a family of related proteins are used to generate a diverse, but focused population of polynucleotides encoding a family of peptide ligands. In another embodiment, the library is a mutagenized population of polynucleotides all derived, for example, from a single parental template. In another embodiment the library is an antibody or ScFv library. In another embodiment the library comprises a polynucleotide population derived from or otherwise related to fibronectin, glutathione S transferase, the immunoglobulin binding proteins such as protein A and protein G and related or derived proteins, trypsin inhibitory peptides and derivatives thereof, thioredoxin, non-antibody members of the immunoglobulin superfamily, lysozyme, or T cell receptors and other appropriate polypeptide scaffolds. In one embodiment, the library is derived by any reiterative diversification and screening techniques. In another embodiment, a plurality of libraries are derived in parallel by methods of reiterative diversification.

The affinity ligand can be a protein, a polypeptide, a peptide, or a fragment of a protein, a polypeptide or a peptide. In one aspect of the invention the ligand is an antibody or an antibody derived polypeptide e.g. an F(ab) fragment, an F(ab′)₂ fragment, or a single chain antibody variable region fragment or scFv. In one embodiment the affinity ligand is a growth factor or hormone or a receptor for a growth factor or hormone (e.g. insulin, tumor necrosis factor, insulin receptor). In another embodiment the affinity ligand is an enzyme and enzyme inhibitor or substrate (e.g. tpA, plasminogen, or fibrin). In yet another embodiment the affinity ligand is a cytokine or a cytokine receptor (e.g. IL-2 or IL-2 receptor). In another embodiment the affinity ligand can be a nucleic acid, a lipid or a sugar. In yet another embodiment the affinity ligand comprises an inorganic molecule or substiuent group.

Depending on the nature of the separation to be done, either the affinity ligand or the protein to which it is known to bind may serve as the affinity ligand. Receptors or binding proteins that recognize gamma-interferon, for example, can provide the starting material to generate a library of potential affinity ligands useful for purifying the hormone itself.

It is sometimes desirable to use a polypeptide receptor as the starting material to develop an affinity ligand. Receptors can frequently be insoluble. Thus, in one embodiment, at least one phenotypic trait which can be enhanced using reiterative diversification can include solubility of a polypeptide receptor or a fragment thereof. A fragment can include a native or natural protein, polypeptide, or peptide, wherein at least one amino acid is removed or missing.

Several classes of protein amenable to such library construction include, but are not limited to the following proteins and/or their receptors IFN α, β, γ, cytokines and/or interleukins (e.g. IL-1, -2, -3, -4), cytokine and/or interleukin receptor antagonists (IL-1ra), colony stimulating factors, erythropoietin (EPO), thymopoietin (TPO), granulocyte stimulating factor (GCSF), granulocyte-macrophage colony stimulating factor (GMCSF), insulin and insulin-like growth factors, blood coagulation factors (Factor VIII, Factor X,), calcitonin, somatostatin, angiotensin I, II, growth factors, growth hormone, transforming growth factors (e.g. TGF α, TGF β), platelet-derived growth factor (PDGF), epidermal growth factor (EGF), growth hormone releasing hormone, luteinizing hormone releasing hormone, growth inhibitory factors (e.g. tumor necrosis factor (TNF)), soluble receptors (soluble TNF receptor (TNF-R), soluble IL-1, IL-2 and other interleukin receptors; insulin binding protein), single-chain antibodies, single-chain antibody dimers and multimers (noncovalent), single-chain antibody dimers and multimers (covalent), monoclonal antibodies, chimeric antibodies, human or humanized monoclonal antibodies, evolved antibodies or antibody fragments, synthetic antibodies, antibodies and/or antibody fragments specific for the protein receptors of: cytokines, growth factors, interleukins, insulin and other polypeptide hormones, lectins and/or adhesion molecules, blood coagulation factors, steroid and thyroid hormones, Dnase, Rnase, tissue plasmingoen activator (tPA), urokinase, streptokinase, therapeutic enzymes.

The affinity ligand can be customized and selected for the biological molecule of interest using reiterative diversification and screening whereby a ligand that binds to the biological molecule of interest can be selected for based upon the desired properties or phenotypic traits of the affinity ligand and the way it interacts with the biological molecule of interest. Thus, a starting ligand might be selected that binds the biological molecule of interest with very high affinity, or lower affinity, and then subsequently optimized and screened for its performance as an affinity separation ligand. An affinity ligand can also be selected that binds the biological molecule of interest within a certain pH range, or under specific salt concentrations, or in the presence or absence of detergents, or in the presence or absence of a chaotropic agent or any other chemically or physically definable condition e.g. the presence or absence of a competitor molecule wherein the competitor molecule is a protein, a polypeptide, or a non-polypeptide “displacer” molecule.

An affinity ligand can also be selected based on its stability profile under various biochemical or chemical conditions. Thus, as examples, but not limitations an affinity ligand can be selected if it displays stability over a specified pH range, at a specified ionic strength, at a specific dielectic constant, in the presence or absence of cations, or at a specific concentration of organic solvents. An affinity ligand can be selected that binds the biological molecule of interest based on any combination of the aforementioned parameters or any other desired chemical or physical parameter.

In one embodiment potential candidate ligands for reiterative diversification and screening can be selected by screening a genetic library derived from any source e.g. an immunized animal, a synthetic expression library, an antibody display library, or bacteriophage or cosmid library. The skilled artisan will recognize there are other sources of genetic diversity that are amenable to reiterative diversification and screening. Techniques for displaying and screening genetic libraries are known in the art. (See e.g. PCT Publication Nos.: WO 01/05808, WO 91/17271, WO 90/02809, U.S. Pat. Nos. 6,174,673, 5,837,500, 6,312,927). The library can include by way of example, but not as a limitation, a phage display library, any eukaryotic cDNA library or an antibody expression library of synthetic or natural origin. The candidate ligand can then be subjected to reiterative diversification to optimize any desired phenotypic traits. The phenotypic traits can include any combination of chemical or biochemical properties of the ligand depending on the specific separation requirements. The ligand can be supplied as one or more resin formulations comprised of a ligand and a solid or semi solid support with the ligand chemically attached to the support through a covalent or noncovalent linker. The resin formulation can be usable in multiple batch purification runs. In one embodiment, the resin is stable based on regenerating conditions or based on criteria defined performance criteria e.g., stability in the presence of sterilizing concentrations of bleach or azide, in the presence of 0.1 N acid or base, and/or temperatures of at least 75° C.

The genetic library of ligands for the biological molecule of interest can be derived from any biological source including but not limited to an animal, a plant, a tissue or organ derived from an animal or plant, a eukaryotic cell or a prokaryotic cell, synthetic polynucleotides or a displayed library of synthetic polypeptides or polynucleotides. Structural and/or functional information stored by electronic means (e.g. a publically available data base) can also be used to design and synthesize targeted libraries for use for multicycle diversification or directed evolution (see e.g., U.S. Pat. Nos. 6,188,965, and 6,269,312).

The library can be screened by expressing the library on the surface of bacteriophage (U.S. Pat. No. 5,223,409, 6,057,103) or using a cell free expression system wherein the library is bound to a solid support (PCT Application No. PCT/GB00/02809). The library can be screened for example for ligand affinity or avidity or stability under various biochemical or chemical conditions. Stability can include, for example, the ability to maintain ligand binding or appropriate conformational expression.

The affinity ligand for the biological molecule of interest can be expressed in any cell known in the art including, but not limited to, any known prokaryotic cell (e.g. E. coli) or any known eukaryotic cell (e.g. Cos cells, HeLa cells, or yeast). Techniques for expressing a ligand with a desired phenotypic trait are known in the art. See Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989). The expressed ligand is purified by any technique known in the art (e.g. ion exchange chromatography, affinity chromatography, or size exclusion chromatography). In one embodiment the ligand is expressed as a fusion protein for ease of purification. As an example, but not a limitation, the fusion protein can be a GST fusion, a His tagged fusion, or a maltose binding protein fusion. After purification the fusion tag can be removed or cleaved. In another embodiment, the fusion tag is not cleaved or removed and the fusion protein is captured directly on a solid support and used for the separation of the molecule of interest. The fusion protein can be captured covalently or non-covalently. In one embodiment, the fusion protein can be coupled to the solid support using a bi-valent cross-linker e.g., 1-2 diamino ethane. A wide range of crosslinking agents are known in the art and can be identified, for example, by consulting the Pierce Biochemicals 2002 product catalog, or other similar sources.

Some fusion partner combinations contemplated in this invention are listed in Table I.

TABLE I

| Affinity Matrices/Ligands | Fusion Binding Domains |
|---|---|
| Metal Chelate matrix | His-Tag |
| 6-His monoclonal antibody | His-Tag |
| Glutathione | GST fusion |
| GST antibody | GST fusion |
| AU1 MAb | AU1 epitope tag |
| AU5 MAb | AU5 epitope tag |
| Amylose | Beta galactosidase fusion |
| Beta-galactosidase | Beta galactosidase fusion |
| Beta-galactosidase MAb | Beta galactosidase fusion |
| c-myc MAb | c-myc epitope tag |
| EE-tag MAb | EYMPME/EFMPME (EE)-tag |
| E-tag MAb | E-tagged scFv Ab |
| Calmodulin | Calmodulin binding peptide fusion |
| Cellulose | Cellulose binding Protein fusion |
| Chitin | Chitin Binding Domain/Intein fusion |
| Dextrin-10 | Maltose Binding Domains |
| Hemagglutinin flu epitope Ab | HA-Tag |
| IgG | Protein A/Protein G |
| S-Protein | S-Tag |

The biological molecule of interest can include a protein, a protein fragment, a fusion protein (e.g. a protein tagged with GST, Fc, Histidine) a pegylated protein, or a peptide. The biological molecule of interest can also include a nucleic acid e.g. DNA, cDNA, a DNA fragment, genomic DNA, RNA, mRNA and cRNA. The biological molecule of interest can also include a lipid e.g. a steroid, a vitamin or a hormone or a receptor for any of the above. The biological molecule of interest can also include a small organic or inorganic molecule e.g. a drug or a prodrug. A small inorganic or organic molecule is one that is less than 50 KD.

The affinity ligand of the biological molecule of interest which has been selected by reiterative diversification can be bound or linked to any solid support known in the art. The affinity ligand can be linked covalently or non-covalently to the solid support. The affinity ligand can be linked to the solid support by ionic interactions, hydrophobic interactions, chemical cross-linking, polymeric coating, or affinity interaction.

Any method known in the art can be used to couple a specific affinity ligand to a solid support. As an example, but not as a limitation the solid support can have an activated carboxyl or aldehyde group on the surface which can react with an amine functional group of the affinity ligand. As another example, but not as a limitation, the solid support can have an epoxy/epoxide group which can react with a thiol or amine of the ligand.

A library of solid supports can be screened in conjunction with a library of potential affinity ligands to optimize a particular phenotypic trait of the ligand. Additional screenings can be done in choosing an appropriate linker chemistry which contributes to the optimization of the desired affinity ligand phenotype.

The solid support can include, but is not limited to a particle, a nanoparticle, a bead, a gel, a membrane or a monolith. The solid support can be comprised of any material which can be covalently or non-covalently linked to the affinity ligand of the biological molecule of interest. Solid supports can be comprised of any synthetic organic polymer such as, but not limited to, polyacrylic, vinyl polymers, acrylate, polymethacrylate, polyacrylamide and nylon. Solid supports can be comprised of a carbohydrate polymer e.g. agarose, cross-linked agarose, cellulose or dextran. Solid supports can also be comprised of inorganic oxides, such as, but not limited to silica, zirconia, titania, ceria, alumina, magnesia (i.e. magnesium oxide), or calcium oxide. Solid supports can also be comprised of combinations of some of the above-mentioned supports such as, but not limited to dextran-acrylamide. Additional examples of solid supports can include multi-tentacle agarose, controlled pore glass, macroporous alumina, divinyl benzene, nitrocellulose, polyacrylates/amides/nylon, polyurethane, metal oxide nanoparticles, polystyrene, polyphenols, polyols, modified polyolefins, polyesters, polylactic acid, diatomaceous earth, or silica. A solid support that minimizes non-specific interactions with the ligand and/or the biological molecule of interest is preferred. The ligand can be directly linked to the solid support or alternatively the solid support can be coated first with a material that enhances binding of the ligand and/or minimizes non-specific interactions with the ligand, the biological molecule of interest or other components from which the biological molecule of interest is to be separated.

In one embodiment the ligand linked to the solid support is contacted with a biological molecule of interest under conditions that promote binding of the molecule of interest to the ligand linked to the solid support. In one embodiment the binding conditions are at physiological pH. In another embodiment binding conditions are at an acidic pH. In another embodiment binding conditions are at a basic pH.

In one embodiment the ligand linked to a solid support is contained within a column. In another embodiment the ligand linked to a solid support is in a buffer solution. The buffer solution can be any solution that does not disrupt the ligand linkage to the solid support or the binding of the molecule of interest to the ligand. In yet another embodiment the ligand linked solid support is on a microarray or chip. In one embodiment, the affinity ligand linked solid support is on a membrane.

In one embodiment the complex of the molecule of interest bound to the affinity ligand linked to the solid support can be washed before elution. Washing is performed to remove non-specifically bound material. The washing step can be performed under process conditions. Process conditions include the conditions used to bind the molecule of interest to the affinity ligand linked to the solid support. In another embodiment the wash conditions can include washing the complex of the molecule of interest and the affinity ligand linked to the solid support with any solution that does not disrupt the affinity ligand linkage to the solid support or the binding of the molecule of interest to the affinity ligand. In another embodiment the wash solution is phosphate-buffered saline (PBS). In another embodiment non-specifically bound material can be removed by centrifugation. In yet another embodiment, the affinity ligand complexed with a molecule of interest can be sorted or collected e.g., using a fluorescent activated cell sorter (FACS) or magnetic beads.

In one embodiment the molecule of interest is eluted or removed from the affinity ligand linked to the solid support by changing the conditions relative to the conditions employed for binding the molecule of interest to the affinity ligand linked to the solid support. The change in conditions can include by way of example, but not as a limitation, a change in pH, a change in salt concentration denaturant, detergent or solute concentration, a change in temperature, or a change in buffer composition.

In another embodiment the invention relates to a composition comprising an affinity ligand for affinity separation linked to a solid support wherein said ligand is produced or selected for by any method of reiterative diversification and screening. The desired phenotype of an affinity ligand can be obtained and/or enhanced using any method of reiterative diversification including but not limited to molecular breeding (see e.g., U.S. Pat. Nos. 5,605,793, 6,319,713, 6,177,263, 6,153,410, 6,117,679, 5,605,793) error prone PCR (Caldwell et al., 1992 PCR Methods Applic., 2: 228), scanning saturation mutagenesis (U.S. Pat. No. 6,180,341), recursive ensemble mutagenesis (Goldman et al., 1992, Nature Biotechnology 10: 1557) and exponential ensemble mutagenesis (Delegrave et al., 1993, Biotechnology Research 11:1548). The ligand may also be mutagenized using a combination of these and other mutagenesis methods. Reiterative diversification can be coupled with any screening or MCAS technology e.g., phage display (U.S. Pat. Nos. 5,223,409, and 5,403,484), or ribosomal display (Mattheakis et al., 1994 Proc. Natl. Acad. Sci. USA 91: 9022) to allow for rapid screening and/or selection of affinity ligands with desired phenotype.

The solid support can include, but is not limited to a bead, a gel, a membrane, a particle a nanoparticle or a monolith. The solid support can be comprised of any material which can be covalently linked to the ligand of the biological molecule of interest (e.g. sepharose, sephadex). Solid supports can be comprised of any synthetic organic polymer such as, but not limited to, polyacrylic, vinyl polymers, acrylate, polymethacrylate, and polyacrylamide. Solid supports can be comprised of a carbohydrate polymer e.g. agarose, cellulose or dextran. Solid supports can be comprised of inorganic oxides, such as, but not limited to silica, zirconia, titania, ceria, alumina, magnesia (i.e. magnesium oxide), or calcium oxide. Solid supports can also be comprised of combinations of some of the above-mentioned supports such as, but not limited to dextran-acrylamide. A solid support that minimizes non-specific interactions with the ligand and/or the biological molecule of interest is preferred. The affinity ligand can be directly linked to the solid support or alternatively the solid support can be coated first with a material that enhances binding of the ligand and/or minimizes non-specific interactions with the affinity ligand, the biological molecule of interest or other components from which the biological molecule of interest is to be separated.

The affinity ligand of the biological molecule of interest which has been selected by reiterative diversification can be linked to any solid support known in the art. The ligand can be linked covalently or non-covalently to the solid support. The affinity ligand can be linked to the solid support by ionic interactions, hydrophobic interactions, chemical cross-linking or polymeric coating.

Any method known in the art can be used to couple a specific ligand to a solid support. As an example, but not as a limitation the solid support can have an activated carboxyl or aldehyde group on the surface which can react with an amine functional group of the ligand. As another example, but not as a limitation, the solid support can have an epoxy/epoxide group which can react with a thiol or amine of the ligand.

In another embodiment the invention relates to a method of isolating an affinity ligand, which possesses a desired phenotype or characteristic, using any method of directed evolution or iterative diversification and screening and linking the affinity ligand to a solid support such that the desired phenotype or characteristic is maintained or enhanced.

The methods of this invention can be used to screen a library of bio-molecules. The library can be obtained from any biological source or can be synthetically generated. The library is screened for affinity ligands with a desired phenotype or bio-chemical or chemical characteristic (e.g. affinity, stability, solubility). Reiterative diversification and screening can be used to optimize the desired trait.(s). The process can be stopped when successive rounds of screening yield no further improvement in the phenotype of the affinity ligand.

Any known method of reiteratve diversification and screening (e.g. molecular breeding, recursive ensemble mutagenesis, error prone PCR, cassette mutagenesis) can be used to obtain the affinity ligand. Once the ligand is obtained it can be cloned and expressed in either a eukaryotic or prokaryotic cell or expressed in an in vitro translation system. Many cloning vectors are known in the art (e.g. pUC). The ligand can be expressed as a fusion protein for ease in purification (e.g. His tagged, GST fusion). Alternatively, the fusion protein comprising the affinity ligand can be linked to a solid support and used directly for affinity separation. After purification the fusion protein can be cleaved to liberate the ligand. The ligand can be purified using any method known in the art. In one embodiment the ligand is purified chromatographically (e.g. ion exchange, size exclusion, affinity chromatography). The purified ligand can be then bound to a solid support using known covalent linkinage chemistry. Alternatively, the affinity ligand can be bound to the solid support via non-covalent linkage (e.g., His Tag Metal chelate matrix combination). The affinity ligand can be screened again for desired phenotypic traits or characteristics and the process repeated such that a solid support and linker chemistry is chosen that optimizes the desired phenotypic trait.

The following examples are offered by way of illustration, and not by way of limitation.

### EXAMPLES 1-11

This example describes the development of a protein ligand for human growth hormone. Discovery and display of affinity ligands capable of binding bovine growth hormone (BGH) have been described in U.S. Pat. No. 5,534,621. Using the methods of the instant invention, one or more ligands capable of binding bovine growth hormone can be systematically altered and improved both for enhanced binding to bovine growth hormone and for binding to human growth hormone (HGH). Moreover, such ligands are further enhanced for their stability in the presence of a solid support and to the conditions associated with immobilization upon such a solid support.

### Example 1

### Methods of Diversification by Enzymatic Fragmentation and Reassembly

Polynucleotides encoding polypeptide ligands capable of binding BGH are obtained, preferably in an existing E. coli expression vector. Alternatively, oligonucleotide primers complementary to 5′-(e.g. encoding the amino terminus of the ligand; Primer: bghlf1) and 3′-(e.g. encoding the carboxy terminus of the binding protein; Primer: bghlr2) are designed and synthesized using methods well known in the art. Both primers are 25 bases in length, hybridizing to the Bovine Growth Hormone Ligand (BGL) templates over 18 bases. The 5′ terminus of the bghlf1 primer encodes the Sfi I restriction enzyme site compatible with the pCANTAB 5E expression phagemid (Amersham-Pharmacia Biotech)(Piscataway, N.J.). The 5′ terminus of the bghlr2 sequence encodes the Not/restriction enzyme site compatible with the 3′ end of the ScFv insertion site on pCANTAB 5e phagemid. The polynucleotides encoding the BGH ligands (BGHL1, BGHL2, BGHL3, . . . ) are amplified and purified using Qiagen Qiaquick PCR Purification Kits(Qiagen, Valencia Calif.), according to manufacturer's instructions. The purified, amplified BGHL products are eluted in 50 uL sterile distilled, deionized water (dd-H₂O).

A. Staggered Extension Process (StEP)

Single-primer PCR reactions are set up as follows. Aliquots of each (n=3) BGHL DNA template are added to a reaction to a final concentration of 10 ng per template (25 pM) in a mixture (50 uL)containing 1×PCR buffer (Perkin-Elmer), 0.125 mM d-NTP (N=A,C,G,T), 1.5 uM MgCl₂, 5 U Taq Polymerase, and one tube with the bghlf primer at a concentration of 1 nM. Identical reactions are set up substituting the bglr1 primer for the the bglf1 primer.

Reactions are subjected to thermocycling procedure designed to allow incomplete extension of the primer from its annealing site to the end of the template. In the instant example the reactions are first heated to 94° C. for 3 minutes, then subjected to 75 cycles of the following thermoprofile (using maximal ramping of heating and cooling temperatures): 15 seconds at 37° C., 30 seconds at 94° C. Following the thermocycling procedure, the samples are rapidly cooled to 4° C.

Samples are purified, and unused primer removed by two cycles of purification over a Qiagen Qiaquick PCR Purification Kits (Qiagen, Valencia Calif.). The sample is eluted each time in 50 uL dd-H₂O. After the second wash, the reaction products are concentrated to <20 uL, and subjected to preparative agarose gel electrophoresis. Polynucleotides with approximate sizes larger than 50 nucleotides, but smaller than the intact template (e.g. <900 bases for a 1000 nucleotide template) are excised from the gel and purified using the Qiagen QIAEX II Gel Extrtaction kit. Product (Qiagen, Valencia Calif.) and eluted into a final volume of 50 uL.

Aliquots (1-10 uL) of the forward and reverse products (e.g. those amplified with the bghlf and bghlr primers, respectively) are combined in a standard PCR reaction and amplified without primer for 10 cycles of the following thermoprofile: 30 seconds at 94° C.; 30 seconds at 55° C.; 60 seconds at 72° C.

Following the initial reaction, bghlf and bghlr primers are added to a final concentration of 10 pM and the samples are amplified through an additional 15 cycles of the same thermoprofile: 30 seconds at 94° C.; 30 seconds at 55° C.; 60 seconds at 72° C.

The randomly reassembled full length products are purified over a Qiagen Qiaquick PCR Purification Kits (Qiagen, Valencia Calif.), digested with Not I and Sfi I, gel purified as described above, and ligated into Not I/Sfi I digested pCANTAB 5E vector. Ligation reactons are set up in 50 uL volumes in 1×OPA+Buffer (Amersham-Pharmacia Biotech)(Piscataway, N.J.), 1 mM ATP, 5 ng/uL pCANTAB 5E vector, 5 U T4 Ligase and 150-200 ng of digested reassembly mixture. Ligation reactions are incubated for one hour at 16° C. The ligase is inactivated by incubating the samples of 70° C. for 10 minutes. The ligated library is transformed into E. coli, infected with M13KO7 M13 helper phage and harvested as a library.

DNase-Based Fragmentation and Reassembly

Multiple double-stranded DNA fragmentation reactions are set up as described in Maniatis, T. Molecular Cloning: A Laboratory Manual. 2nd, ed., Cold Spring Harbor Laboratory, Cold Spring Harbor Laboratory Press, Cold Spring Harbor, N.Y., 1989 and modified by U.S. Pat. No. 5,605,793 in reaction mixtures containing 1 uL of the sample DNA, 0.1, 0.01, and 0.001 units of DNase I. Following brief incubations at room temperature, the reactions are stopped and the reaction products separated on a preparative agarose gel. The digested templates strands are visualized by DNA staining as ‘smear’ extending from about 50 bases to a molecular weight approaching that of the undigested templates.

DNA fragments between 50 and 250 bp are excised from those lanes in which smears are visible in this range. The fragments are excised from the gel and purified using the Qiagen QIAEX II Gel Extrtaction kit (Qiagen, Valencia Calif.). The product is eluted in a final volume of 50 uL.

Equivalent aliquots (5-10 uL) of each fragmentation product are reassembled in a standard PCR reaction mixture and amplified without primer for 25 cycles of the following thermoprofile: 30 seconds at 94° C.; 30 seconds at 55° C.; 60 seconds at 72° C.

Following these 25 cycles, bghlf and bghlr primers are added to a final concentration of 10 pM and the samples are amplified through an additional 15 cycles of the same thermoprofile: 60 seconds at about 94° C.; 30 seconds at 55° C.; 60 seconds at 72° C.

The randomly reassembled full length products are purified over a Qiagen Qiaquick PCR Purification Kits (Qiagen, Valencia Calif.), digested with Not I and Sfi I, gel purified as described above, and ligated in into Not I/Sfi I digested pCANTAB 5E vector. Ligation reactons are set up in 50 uL volumes in 1×OPA+Buffer (Amersham-Pharmacia Biotech), (Piscataway, N.J.) 1 mM ATP, 5 ng/uL pCANTAB 5E vector, 5 U T4 Ligase and 150-200 ng of digested reassembly mixture. Ligation reactions are incubated for 1 hr at 16° C. Ligase is inactivated by incubating the samples of 70° C. for 10 minutes. The ligated library is transformed into E. coli, infected with helper phage and harvested as a library.

### Example 2

### Expression and Rescue of Primary Ligand Libraries

Electroporation competent TG1 cells are prepared according to established protocols. Briefly, 1 ml of freshly prepared competent E. coli TG1 cells are added to prechilled, 50 ml sterile disposable centrifuge tubes and placed on ice. Approximately, 25-50 uL of each ligation reaction (or uncut or other controls) is added to a separate aliquot of the TG1 cells. The mixture is swirled gently and incubated on ice for 45 minutes. The tubes are then transferred to a 42° C. water bath and incubated for two minutes, then placed briefly back on ice. For each library, the undiluted, transformed TG1 cells are incubated for one hour at 37° C., plated in 50 uL aliquots on 18 separate SOBAG plates (SOBAG: 20 g/L Bacto-tryptone, 5 μL Bacto-yeast extract, 0.5 g/L NaCl, 10 mM MgCl₂, 110 mM glucose, 100 ug/mL ampicillin; Plates 1.5% Bacto-agar). Plates are inverted and incubated 20 hours at 30° C. Phage are harvested by flooding each plate with 5 ml 2×YT medium and scraping cells with a sterile glass spreader.

Phage libraries are rescued by diluting cells to an A600 of 0.3 in 2×YT medium. Ampicillin is added to a final concentration of 100 ug/mL, and glucose to a final concentration of 2%. The cultures are incubated for one hour at 37° C. with mild shaking (250-300 rpm).

Aliquots of prepared M13KO7 bacteriophage are added at a multiplicity of infection of about 5:1, and the cultures are incubated for an additional one hour at 37° C. with mild shaking.

Cells are sedimented by centrifugation at 1000×g for 10 minutes, resuspended in 10 ml 2×YT medium containing 100 ug/ml ampicillin and 50 ug/ml kanamycin (2×YT-AK), and incubated overnight at 37 C with mild shaking. Cells are again sedimented at 1000×g for 20 minutes. The supernatant is harvested, transferred to a sterile polypropylene centrifuge tube and stored at 4° C.

### Example 3

### Library Screening and Ligand Rescue

Two (2) ml PEG-NaCl solution is added to each of the phage-containing supernatants (PEG-NaCl: 20% PEG 8000, 2.5 M NaCl in distilled water) and incubated on ice for 60 minutes. Phage are pelleted by centrifugation at 10,000×g at 4° C. for 20 minutes. Supernatant is thoroughly removed and discarded. Tissue flasks (25 cm³) are coated with human growth homone (HGH) by diluting commercially available HGH peptide to 10 ug/mL in PBS and incubating at room temperature for 2 hours. Flasks are washed 3 times with PBS, then treated with blocking buffer (blocking buffer: PBS with 1% nonfat dry milk). Flasks are incubated with blocking buffer for 1 hour at room temperature. 14 ml of blocking buffer containing 0.01% thimerosal is added to the (16 ml) bacteriophage suspensions and incubated at room temperature for 15-20 minutes. The diluted phage library (20 ml) are added to each of the corresponding flasks and incubated for two hours at 37° C. The flasks are emptied and washed 20 times with PBS (20 ml), followed by an additional 20 washes with PBS containing 0.1% Tween 20.

At this point, the library may be further enriched or tuned by reinfecting log-phase TG1 cells with the flask-adherant phage population. The subsequent selection steps may also include increasingly stringent and increasingly selective criteria related to either the physicochemical, chemoselectivity, or physical or chemical stability properties of the ligands. For example, subsequent cycles of screening might employ higher or lower temperatures, salt concentrations, trace organic solvents, simulated shear forces, and other conditions that might eliminate ligands with properties incompatible with the intended process applications. For affinity ligand development it is important early in the screening process to focus the binding and release properties in the range required for effective adsorption and product recovery. Typically, this means that that the ligand will bind the target protein with high selectivity, but an affinity not exceeding 1×10⁻⁹ M⁻¹ under binding conditions, and release >95% of the ligand under elution conditions. Preferred elution conditions will involve moderate changes in pH, dielectric constant, temperature, solvent denaturant or competitor concentrations.

In the present example, the bound phage are allowed to reinfect E. coli TG1 cells directly in the adsorption flask by adding 10 mL of log-phase TG1 cells to the flask and incubating at 37° C. for one hour with mild shaking. The entire culture is then transferred to disposable polypropylene centrifuge tube.

Aliquots (100 uL) of the infected (phagemid-containing) cells are spread onto a series of SOBAG agar plates using a sterile spreader. Plates are inverted and incubated at 30° C. for 20 hours. Individual colonies are picked and grown overnight in SOBAG media (5 ml) at 30° C. Phagemid DNA is prepared from the overnight cultures using standard methods (e.g. QiaPrepSpin Miniprep kits) (Qiagen, Valencia Calif.). Phagemid DNA is digested with Sfi I and Not I restriction enzymes, and the insert size determined by agarose gel electrophoresis (1% agarose). Inserts migrating at a size similar to that expected of the original BGHL are gel purified.

### Example 4

### Second Round Library Generation and Chemoselection

Generation of a second round library can be accomplished by any of the multi-cycle diversity generation methods described in the specification. In the instant example, insert DNA is prepared from each of the inserts from Example 3 having the appropriate insert size. Insert DNA is mutagenized and recombined using diversity generation methods described in Example 1. The libraries prepared in this manner are expressed and rescued as described in Example 2. In this example, the objective is to develop an affinity resin suitable for isolating HGH from a culture of Chinese Hamster Ovary (CHO) cells stably transformed with a construct encoding the constitutive expression of the HGH gene, under the control of the cytomegalovirus promoter. To select for HGH ligands with specificity for HGH but not BGH or components in CHO lysates, the screening of the Second Round libraries proceeds via a modified version of that in Example 3. Following Step 2 of Example 3 two selection flasks are prepared for each library.

The first flask is the nonspecific adsorption flask. It is treated with CHO lysate at room temperature for two hours. CHO Lysate is prepared as follows: 1-150 mm plate of confluent CHO cells are scraped in 10 ml PBS, collected in 50 ml disposable, sterile centrifuge tubes, sonicated using standard equipment and methods, sedimented by centrifugation at 10,000×g for 20 minutes. The supernatant is collected and diluted to 30 ml. Diluted supernatant is the CHO Lysate.

The second flask is treated with HGH by diluting commercially available HGH peptide to 10 ug/mL in PBS and incubating for two hours at room temperature. The flasks are washed three times with PBS, then treated with blocking buffer for 1 hour at room temperature. Blocking buffer (14 ml) containing 0.01% thimerosal is added to the (16 ml) bacteriophage suspensions and incubated at room temperature for 15-20 minutes. The diluted 2^{nd} round phage libraries (20 ml) are first added to the CHO Lysate treated flasks and incubated for two hours at 37° C.

Following adsorption to the CHO Lysate coated flasks, the diluted libraries are removed. To eliminate binders with high affinity to both HGH and BGH, BGH (to a final concentration of 1×10⁻⁸ M) is added directly to the phage suspensions and the suspensions transferred directly to the first and second selection flasks described above. The flasks are again incubated for two hours at 37° C. The flasks are emptied and washed 20 times with PBS (20 ml), followed by an additional 20 washes with PBS containing 0.1% Tween 20.

The bound phage are allowed to reinfect E. coli TG1 cells directly in the adsorption flask by adding 10 mL of log-phase TG1 cells to the flask and incubating at 37° C. for one hour with mild shaking. The entire culture is then transferred to a disposable polypropylene centrifuge tube.

Aliquots (100 uL) of the infected (phagemid-containing) cells are spread onto a series of SOBAG agar plates using a sterile spreader. Plates are inverted and incubated at 30° C. for 20 hours. Individual colonies are picked and grown overnight in SOBAG media (5 ml) at 30° C. Phagemid DNA is prepared from the overnight cultures using standard methods (e.g. QiaPrepSpin Miniprep kits (Qiagen, Valencia Calif.). Phagemid DNA is digested with Sfi I and Not I restriction enzymes, and the insert size determined by agarose gel electrophoresis (1% agarose). Inserts migrating at a size similar to that expected of the original BGHL are gel purified.

### Example 5

### Library Arraying and Quantitation

Individual colonies picked in Example 4 are arrayed both on a SOBAG plate (for storage) and are inoculated into a single well of a 96 well microtiter plate containing 400 uL 2×YT-AG medium [2×YT-AG: 2×YT medium with 100 ug/ml ampicillin and 2% glucose]. The plates are incubated overnight at 30° C. with shaking.

Soluble antibodies are prepared from the samples grown on the microtiter plates, and the presence and relative concentration of antibody in each well is determined using the Anti E Tag ELISA methods described in the Recombinant Phage Antibody System™ (RPAS) define instruction manual (Amersham-Pharmacia) (Piscataway, N.J.).

Additional soluble protein (or phage antibody) can be prepared for subsequent screening by duplicating the methods described here and in the Amersham-Pharmacia instruction manual.

### Example 6

### Test for Stability Under Noncovalent Immobilization Conditions

In this example, the Anti E Tag ELISA method (see Example 5) is modified slightly to test for the stability of the binding activity when each of the positive ScFv antibodies identified in Example 5 is exposed to a solid surface (polystyrene) for an extended period (overnight). This example allows for rapid elimination of antibodies likely to be unstable when immobilized by any means.

Duplicate microtiter plates are coated with Anti Tag E antibodies for 1-2 hours at room temperature, washed, and blocked with blocking buffer essentially as in Example 5.

Duplicate aliquots of the soluble antibody samples testing positive in Example 5 are added to and incubated in the Anti E Tag wells for two hours at room temperature.

The wells are washed several times with PBS, followed by addition and incubation overnight in blocking buffer at room temperature.

Binding of HGH to the immobilized antibodies is tested using commercially available biotin-HGH and standard streptavidin-alkaline phosphatase ELISA development methods.

Duplicate wells that demonstrate little or no signal following this incubation sequence encode ligands likely to be unstable upon immobilization and they are eliminated from subsequent rounds of ligand screening. Note, however, that such “negatives” can provide an important source of sequence diversity or sequence information for subsequent rounds of diversity generation.

### Example 7

### Testing for Compatibility with Epoxide, CnBr, Activated Carboxylate, Activated Imine, Imide, and Amine Linkages

In this example, the candidate ligands identified in Examples 5 or 6 are tested for stability in the presence of a variety of activated ligands (e.g. linkers) most commonly used for covalent immobilization of affinity ligands. Two methods can be used to rapidly test ligands for linker compatibility. The first is to modify the ELISA process of Example 5 by preparing a series of identical wells from each of the supernatants by coating the wells with the supernatants, blocking, and then exposing individual wells in each set to a different activated support or linker, followed by development of the well with Anti E Tag antibody. The second, and milder strategy is to prepare a series of identical wells from each of the positive clones from Examples 5 or 6, using the Anti E Tag antibody to capture the candidate ligands (as in Example 6). Following capture, as in Example 6, Wells are blocked, and individual wells in each set are exposed to a different activated support (e.g. 1-2 hour at room temperature). Following a one hour incubation at room temperature, the wells are developed by sequential incubation with biotin-HGH followed by streptavidin-alkaline phosphotase as described in Example 6.

Since agarose is one of the milder support matrices one can use for affinity chromatograpy, distinct linker chemistries are tested within the context of an agarose matrix. The following activated agarose resins are tested with each of the ligand candidates.

Resin Product (Sooner, Inc) (Garvin, OK) Coupling Chemistry

| | |
|---|---|
| ACG-4 Affarose G | Periodate-activated hydroxl |
| ACG-6 Affarose G | Activated Amine |
| ACA-4 Affarose AE | ω-Aminohexyl |
| ACA-6 Affarose AE | Activated Carboxylate |
| ACA-6 Affarose AE | Diaminopropyl |
| ACA-6 Affarose AE | Adipic Acid Hydrazide |
| ACT-4 Affarose T | Thiol Propyl |

In each case, slurries containing the activated agarose product (50-75 uL) are added to microwells containing 200 uL PBS, and the microtiter plates containing candidate ligands, are incubated at room temperature for 1-2 hours, washed with PBS and developed using biotin labeled HGH. Wells retaining binding activity are considered to be active. Those with the highest amount of retained activity are identified as preferred coupling agents.

These ligands can also be tested for retention of biological (e.g. binding) activity following coupling of the candidates to any one of these or other immobilization supports. Instructions for coupling proteins to the supports are provided by the manufacturer.

### Example 8

### Testing for Compatibility with a Variety of Solid Supports

Using methods analogous to those described in Example 7, a series of coupling (e.g. immobilization) reactions are set up with each of the candidates in the ligand libraries from Examples 5 or 6. Candidate ligands are coated or captured onto a series of identical microtiter wells as described above. Commercially purchased samples of the following support materials are added to the series of microwell corresponding to each candidate ligand:

| Particle/Bead Material | Product(s)/(Manufacturer(s)) |
|---|---|
| Agarose | Sepharose ™ (e.g. Amersham- |
| | Pharmacia, Piscataway, NJ) |
| Crosslinked Agarose | Sephacryl ™ (e.g. Amersham- |
| | Pharmacia, Piscataway, NJ) |
| Multi-tentacle Agarose | Tentacle ™ Gels (e.g. Tosohaus or |
| | Tosoh Bioscience, LLC) |
| | Montgomeryville, PA.) |
| Controlled Pore Glass | (Prime Synthesis, Inc. (Astor, PA) and |
| | CPG, Inc. (Lincoln Park, NJ)) |
| Macroporous Alumina | (UOP) |
| Divinyl Benzene | (BioRad Laboratories, Inc) (Hercules |
| | CA) |
| Cellulose | (Whatman, Inc.) (Clifton, NJ) |
| Nitrocellulose | (Whatman, Inc) (Clifton, NJ) |
| Nylon beads | Nylon 6,6 (DuPont) (Wilmington, DE) |
| Metal oxide nanoparticles | Silicon dioxide beads (Cabot |
| | Corporation) (Billerica, MA) |

Development of wells following exposure of ligands to the above supports is done as in Example 7.

### Example 9

### Additional Multicycle Diversity Generation and Screening for Focused Improvement

Candidate ligands testing positive in one or more chemoselection screens and one or more physical stability screens are subjected to an additional round of reiterative diversification using the methods discussed in Example 1. Subsequent selection and screening is done as in previous examples except that chemoselection tests the capacity of the ligand to both bind and release the target species in response to a change in ionic strength, pH, temperature, or the addition or change in concentration of a solvent, denaturant, chelator, competitor, displacer or other chemical entity whose presence is for the purpose of reducing or eliminating the interaction between the ligand and its target.

### Example 11

### Further Improvements in the Ligand and the Process

It will be understood by one of skill in the art that the steps outlined in the examples above are illustrative and can be optimized and accelerated by a number of means. For examples, many of the incubations that take place over several hours can be shortened substantially, with only minimal impact on overall efficiency. Likewise, phage rescue and soluble antibody (phagemid expression) testing can be done concommitantly at both the library and clonal levels to accelerate the process. Moreover, many of the operations described herein can be automated by one of skill in the art. In addition, clones may be sequenced and characterized after any of the isolation steps described herein and the information contained in such operations used for the generation of focused libraries using site-directed approaches to multi-cyclic diversity generation. The ligand can be improved using additional rounds of reiterative diversification. Additional rounds can select for ligands with binding affinities of <10⁻⁹ M⁻¹>10⁻⁶ M⁻¹ and are expressed at a concentration of at least 20 g/L (preferably 100 mg/L) and stable in the presence of sterilizing concentrations of bleach.

### Example 12

### Generation of Anti-Insulin ScFv Ligands For Purifying Human Insulin

Monoclonal antibodies to insulin are known in the literature, as is the methodology required to prepare a ScFv fragment from polynucleotides encoding a full length antibody. The paper by Lake et al. 1994, Mol. Immunology 31(11):845, described the generation of one such antibody. This antibody, and others generated from anti-insulin hybridomas cDNA are unsuitable in both affinity and stability for use in affinity chromatography. To generate improved anti-insulin affinity resins, the ScFv(s) described here are subjected to multi-cycle diversity generation and subjected to a sequence of screening steps analogous to Examples ______. The screening sequence systematically enhances and defines the performance attributes of the resulting single-chain antibody such that the antibody(s) derived from the multi-cycle diversity generation and screening process are suitable for production, immobilization and use in affinity chromatography.

Following the ligation of sequences of interest into the pCANTAB 5E vector, the key steps involved in developing a phage display library are described below. Competent E. coli TG1 cells are transformed and grown as described in Example 3. The library is rescued (e.g. phagemid population) by infecting transformed TG1 cells with helper phage M13KO7.

The phage population is expanded by infecting new TG1 cells and concentrated, via PEG-based precipitation (e.g. with PEG 8000, or similar products).

The phage are adsorbed to a surface, magnetic bead, particle or other matrix coated with insulin. Typically, the conditions for this adsorption reflect the minimal binding properties desired in the product. Bacteriophage that do not adhere under the desired conditions, are washed away by multiple high stringency washing steps. Stringency can be adjusted by changing the salt concentration or organic solvent concentration. High stringency wash steps include washing with organic solvents at concentrations >0.1% and/or in salt concentrations less than 100 mM or greater than 200 mM.

Adsorbed phage are rescued in one of several ways. First, they can be released from the adsorption surface by high or low ionic strength wash or the addition of competitor (e.g. target) protein. Following elution, the selected phage are re-concentrated using PEG. Second, and more typically, infection-ready TG1 (e.g. log phase) cells are added directly to the adsorption flask (particles or surface), and the adsorbed phage are allowed to directly infect the TG1 cells and expand in population for several hours.

The rescued recombinant phage are then allowed to infect the HB2151 such that they allow formation of individual colonies on an ampicillin containing nutrient agar plate.

Individual colonies can be tested for their binding to Anti-E Tag Antibody to confirm the presence of an expressed fusion protein.

All references cited herein are incorporated herein by reference in their entirety and for all purposes to the same extent as if each individual publication or patent or patent application was specifically and individually indicated to be incorporated by reference in its entirety for all purposes. To the extent publications and patents or patent applications incorporated by reference contradict the disclosure contained in the specification, the specification is intended to supercede and/or take precedence over any such contradictory material.

All numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Many modifications and variations of this invention can be made without departing from its spirit and scope, as will be apparent to those skilled in the art. The specific embodiments described herein are offered by way of example only and are not meant to be limiting in any way.

What is claimed is:

1. A method of isolating an affinity ligand for affinity separation of a molecule of interest comprising the following steps:
a) selecting a starting ligand;
b) altering the starting ligand using a process of reiterative diversification;
c) isolating the altered ligand of b);
d) screening the isolated ligand of c) for the presence of a desired phenotype;
e) optionally repeating any of steps b), c) and d);
f) chemically linking the ligand of d) or e) to a solid support.

2. The method of claim 1, wherein the desired phenotype is ligand affinity for the molecule of interest.

3. The method of claim 1, wherein the desired phenotype is enhanced ligand stability compared to the starting ligand.

4. The method of claim 2, wherein the ligand affinity for the molecule of interest is enhanced as compared to the starting ligand.

5. The method of claim 1, wherein the starting ligand is a protein or a protein fragment.

6. The method of claim 1, wherein the starting ligand is an antibody or an antibody fragment.

7. The method of claim 6, wherein the antibody fragment is an Fab or an F(ab′)₂.

8. A composition comprising an affinity ligand chemically linked to a solid support wherein said affinity ligand is obtained by a process of reiterative diversification.

9. The composition of claim 8, wherein the affinity ligand is a protein or a protein fragment.

10. The composition of claim 8, wherein the affinity ligand is an antibody or an antibody fragment.

11. The composition of claim 10, wherein the antibody fragment is an Fab or an F(ab′)₂.

12. A method of isolating a molecule of interest using a custom designed affinity ligand comprising the following steps:
a) obtaining a genetic library which encodes a candidate affinity ligand for the molecule of interest;
b) altering the genetic library using a method of reiterative diversification such that the affinity ligand expresses a desired phenotype;
c) expressing the altered genetic library of b);
d) screening the altered library for an affinity ligand for the molecule of interest wherein said altered affinity ligand expresses the desired phenotype;
e) optionally repeating any of b), c) and/or d);
f) isolating the affinity ligand after any of steps c), d), or e);
g) chemically linking the isolated affinity ligand of f) to a solid support;
h) contacting the affinity ligand linked to the solid support with the molecule of interest under a first set of conditions such that the molecule of interest binds to the affinity ligand of g);
i) altering the first set of conditions of h) such that the molecule of interest separates from the affinity ligand linked to the solid support.

13. The method of claim 12, wherein the solid support is a bead.

14. The method of claim 12, wherein the solid support is a gel.

15. The method of claim 12, wherein the solid support is a microarray or chip.

16. The method of claim 12, wherein the solid support is a membrane.

17. The method of claim 12, wherein the affinity ligand is a protein or protein fragment.

18. The method of claim 12, wherein the affinity ligand an antibody or antibody fragment.

19. The method of claim 18, wherein the antibody fragment is an fab or an F(ab′)₂.

20. The method of claim 12, wherein the desired phenotype is increased affinity of the affinity ligand for the molecule of interest compared to the affinity of the affinity ligand candidate encoded by the genetic library for the molecule of interest.
